(19) United States
(12) Patent Application Publication
(10) Pub. No.: US 2025/0381076 A1
(43) Pub. Date: Dec. 18, 2025

(54) Absorbent Article with Adhesive Patches

(71) Applicant: Essity Hygiene and Health Aktiebolag, Goteborg (SE)

(72) Inventors: Josefin Sohl, Göteborg (SE); Mateo Morales, Göteborg (SE); Fabio Lopez, Medellin (CO)

(21) Appl. No.: 18/879,086

(22) PCT Filed: Jul. 11, 2022
(86) PCT No.: PCT/EP2022/069219

§ 371 Date: Dec. 26, 2024

(51) Int. Cl. A61F 13/472; A61F 13/15; A61F 13/56

(52) U.S. Cl. CPC A61F 13/472; A61F 13/15699; A61F 13/5616

(57) ABSTRACT

The disclosure relates to an absorbent article having a crotch portion comprising first and second front wings that extend outwardly from the crotch portion in a transverse direction (x) and a rear portion comprising first and second rear flaps that extend outwardly from the rear portion in the transverse direction (x). The first and second front wings each comprise respective first and second crotch adhesive patches, wherein the crotch adhesive patches are offset from each other in the longitudinal direction (y) by an offset distance dO, and the first and second rear flaps each comprise respective first and second rear adhesive patches, wherein the rear adhesive patches are also offset from each other in the longitudinal direction (y) by the same offset distance. Also disclosed is a method for manufacturing an absorbent article with offset adhesive patches.

### CROSS REFERENCE TO RELATED APPLICATIONS

This application is a National Stage application of PCT/EP2022/069219, filed Jul. 11, 2022, the contents of which are incorporated by reference in their entirety herein.

### TECHNICAL FIELD

The present disclosure relates to an absorbent article of the type having a front portion, a rear portion, and a crotch portion arranged between the front and rear portions, with wings that extend outwardly from the crotch portion for affixing the article to a garment of a user. Examples of such articles may include incontinence articles, sanitary towels, panty liners, veils and the like. The disclosure also relates to a method of manufacture of such articles.

### BACKGROUND

The general purpose of such absorbent articles is to absorb, distribute and store various types of body exudates while providing a high level of comfort and sense of dryness to the wearer during use of the absorbent article. Also, such absorbent articles are arranged to prevent the wearer from getting the clothes soiled by body exudates.

A conventional sanitary napkin is normally designed with a main body which includes a liquid-permeable topsheet, a liquid-impermeable backsheet and an absorbent core which is sandwiched between the topsheet and the backsheet. The main body is arranged along a longitudinal axis and a transversal axis which extends in a perpendicular direction in relation to the longitudinal axis.

Furthermore, absorbent articles in the form of sanitary napkins are often provided with two wings, i.e. two wing-shaped fastening elements, extending outwardly from the main body in opposite directions and generally along the transversal axis. The purpose of the wings is to allow fastening of the sanitary napkin to an undergarment of a user. To this end, the wings are arranged to be folded over the main body and towards said longitudinal axis. In order to allow fastening of the sanitary napkin to the undergarment, each wing is provided with fastening means such as an adhesive material or hook and eye type fasteners. In this manner, the sanitary napkin can be attached and held in place in the undergarment during use.

According to the prior art, the wings of a sanitary napkin can be formed in a symmetrical manner, i.e. so that the entire sanitary napkin can be said to define a contour which is symmetrical with reference to its longitudinal axis. Alternatively, it is also known that the wings can be formed in an asymmetrical manner with reference to the longitudinal axis.

A previously known absorbent article having asymmetrical wings of the above-mentioned type is known from the patent document WO 2019/007527. This document shows a sanitary napkin having two wings which are arranged asymmetrically along opposite sides of a longitudinal edge of the napkin.

In certain cases, it is desirable to have further wings or flaps that can provide further opportunity to fasten the article to a garment. This is particularly the case for articles that extend beyond the crotch region of a user and have a considerable forward or rearward extension. In the case of articles to be used during the night, the further flaps may be provided at the rear and used to adhere to or fold around the seat of an undergarment. Such rear flaps or hip flaps can also be fastened with similar means such as adhesive patches. An example of an absorbent article having both front wings and rear wings is shown in US2004/0243087. In this case, adhesive patches are provided on all four wings. The adhesive patches are spaced in a rectangular configuration, with both left and right pairs of patches being equally spaced in the longitudinal direction and both front and rear patches being equally spaced in the transverse direction.

Manufacture of such an article may of course be highly automated in order to ensure cost-effective and consistent products. Conventional production takes place using continuous topsheet and backsheet webs of material to which the core is applied to form a precursor web. The adhesive patches are generally applied, using a slot coating procedure, onto a carrier web such as a siliconised release paper. They are subsequently applied or transferred to the precursor web, from which the individual absorbent articles can finally be cut. The carrier web may be in the form of a single bridging web, carrying adhesive patches for both the left and the right side wings. Alternatively, individual (narrower) carrier webs may be provided for the respective left and right side wings. This requires less material usage.

For previous arrangements with four wings, the respective adhesive patches have been spaced in a rectangular configuration, with both left and right pairs of patches located at the same respective longitudinal position and being equally spaced in the longitudinal direction and furthermore with both front and rear patches being equally spaced in the transverse direction. This configuration has been considered important for smooth operation of the production process. For a single bridging web, the positions of the glue patches are fixed with respect to each other and can only be adjusted by changes to the slot coating procedure. In general, it is preferable to have just one left side slot applicator and one right side slot applicator so the front and rear patches are aligned. Furthemore, if individual left and right carrier webs are used, the front and rear patches are also aligned i.e. equally spaced from the longitudinal axis. Nevertheless, even if individual left and right carrier webs are used, it remains desirable and important for both webs to be mounted on a common shaft in order to ensure that the timing accuracy is achieved. This then means both carrier webs will travel at the same speed, which determines that the separation between the front and rear patches in the longitudinal direction for the respective left and right sides will be the same.

Although the above fixed rectangular configuration is generally acceptable for many absorbent articles, it is unable to adequately be adapted to articles where certain asymmetry is required. Consequently, there is a need for further improvements to articles and processes.

### SUMMARY

According to the invention, there is disclosed an absorbent article having a crotch portion including first and second front wings that extend outwardly from the crotch portion in a transverse direction (x) and a rear portion including first and second rear flaps that extend outwardly from the rear portion in the transverse direction (x). The first and second front wings each include respective first and second crotch adhesive patches, wherein the crotch adhesive patches are offset from each other in the longitudinal direction (y) by an offset distance dO, and the first and second rear flaps each include respective first and second rear adhesive patches, wherein the rear adhesive patches are also offset from each other in the longitudinal direction (y) by the same offset distance. In this context, the rear flaps are referred to as flaps in order to distinguish them from the wings, located at the crotch. Nevertheless, it will be understood that the terms wings and flaps may be used interchangeably. Furthermore, reference is given to first and second front wings which are located on respective left and right sides of the article. These may also be referred to as left and right wings respectively, with reference to their position with respect to a body of a user when in use. The same nomenclature may be used for e.g. the first and second rear flaps and other parts of the article.
a. The absorbent article may be any appropriate item that is intended to be affixed within a garment for absorbent purposes. This may include incontinence articles, sanitary towels, panty liners, veils and the like. The article may be of otherwise conventional construction. In general, such articles include a liquid-permeable topsheet, a liquid-impermeable backsheet, an absorbent core arranged between the topsheet and the backsheet, with a central transverse axis (X) and a central longitudinal axis (Y). The resulting absorbent article includes a front portion, a rear portion and a crotch portion arranged between the front and rear portions with a length of the absorbent article in the longitudinal direction (y) being defined between a front edge of the front portion and a rear edge of the rear portion at the central longitudinal axis. In embodiments, the length of the article in the longitudinal direction may be in a range from 150 mm to 400 mm, preferably from 280 mm to 327 mm. Furthermore, the front and crotch portions may each include 30% of a total length of the absorbent article in the longitudinal direction (y), and the rear portion may include 40% of the total length.

As a result of the fact that the offset distances are equal, a repeat distance (dA) between the first crotch adhesive patch and the first rear adhesive patch in the longitudinal direction (y) is equal to a repeat distance (dB) between the second crotch adhesive patch and the second rear adhesive patch in the longitudinal direction (y). In this context, the repeat distance is measured from the front edge of the respective adhesive patches. It will be understood that if both patches are the same size, then the repeat distance will be the same wherever it is measured. By ensuring that both the left and right pairs of patches have the same spacing, the placement of the adhesive patches during the manufacturing stages can be simplified. This applies both when the left and right patches are applied to a common carrier web or where separate left and right carrier webs are used, mounted to a common roller or axle.

As a result of the offset between the first and second crotch adhesive patches, a greater length of adhesion is achieved in the crotch region, since, going from front to rear, the absorbent article is first adhered at the left side and then adhered at the right side (or vice-versa). The offset between the patches can also help ensure that when folding the wings or flaps, the adhesive patches do not become attached to each other. Other advantages can be present for offset rear patches too, due to their asymmetric placement with respect to the surface of an undergarment. It will be understood that in addition to the adhesive patches located on the wings and the flaps, additional adhesive regions or patches may be provided elsewhere on the back sheet for connection to an undergarment.

The repeat distance will depend on the individual dimensions and shape of the absorbent article. In certain embodiments, the distances (dA, dB) are in a range from 100 mm to 250 mm, preferably from 140 mm to 200 mm.

The offset distance will also depend on the individual dimensions and shape of the absorbent article. In certain embodiments, the offset distance dO is in a range of from 10 mm to 40 mm, preferably between 15 mm and 30 mm, more preferably around 20 mm.

The absorbent article may have any appropriate outer contour, with symmetry about either the longitudinal axis or the transverse axis, both or neither. The invention is particularly applicable to asymmetric articles, especially those having asymmetry about the longitudinal axis. In this manner, the offset between the left and right side adhesive patches can be used to better align to the asymmetry of the outer contour. In one embodiment, the first and second front wings extend outwardly from the crotch portion in an asymmetric manner with respect to the central longitudinal axis (Y). This means that the first and second front wings are also offset with respect to each other along this axis i.e. the first front wing may be located more forwardly than the second front wing (or vice-versa). Each wing may also be asymmetric with respect to the central transverse axis (X).

Also the first and second rear flaps may have any appropriate shape and may be symmetrical or asymmetrical. In a particular embodiment, the first and second rear flaps are symmetrical with respect to the central longitudinal axis (Y), while the first and second front wings are asymmetrical. In other words, a distance between the left front wing and rear flap will be different to the distance between right front wing and rear flap. This leads to an overall construction of the article, whereby all of the adhesive patches and the front wings are offset at the respective left and right sides of the article, while the rear flaps are not. The rear adhesive patches will thus be located at different relative positions on the respective rear flap. As the rear flaps are generally larger in area than the front wings, there is more area onto which the rear adhesive patches can be acceptably attached, without approaching an edge of the article. Thus the manufacturing benefits of having equal offsets between the respective left and right adhesive patches can be realised even though the rear flaps are not offset.

It will be appreciated that the offset between the left and right side adhesive patches may be different to the offset between the left and right front wings. The front adhesive patches may thus also be located at different relative positions on the respective front wings.

As noted above, the absorbent article may have any appropriate construction conventionally applicable for such articles. In an embodiment, the absorbent core has a smaller extension in a plane of the longitudinal and transverse axes than an extension of the backsheet and/or topsheet. The body facing surface of the backsheet can then preferably be joined at a perimeter to the garment facing surface of the topsheet. In an embodiment, the topsheet can have substantially the same extension in a plane of the longitudinal and transverse extension of the absorbent article and edge areas of the absorbent article are joined together. It is not however excluded that the backsheet might have a larger extension than the topsheet or vice versa.

The topsheet and the backsheet may be joined by conventional means, depending on the chosen materials. In particular, they may be joined by adhesives, heat sealing, embossing and/or ultrasonic processing.

The absorbent article may further include a liquid acquisition sheet arranged between the topsheet and the absorbent core. Such acquisition layers are generally known and can in particular be adapted to quickly receive and temporarily store discharged liquid before it is absorbed by the absorbent core

According to the present disclosure, the adhesive patches may be of any appropriate type, suitable for the intended function of releasable attachment to a user's garment without damaging or otherwise marking the garment. The term ‘adhesive’ is not intended to be limited to glues but may also encompass other adherent materials such as hook-like microfasteners. Preferred adhesive materials are those that can be applied as a coating using a slot-coating applicator as presently installed in existing production lines. These include pressure sensitive adhesives including cold press adhesives. The adhesive patches may also be deposited by any other suitable means, including jet application, delivery nozzle, transfer and the like.

The adhesive patches may have any particular form depending on the method of application. In an embodiment, the crotch adhesive patches and the rear adhesive patches are generally rectangular in shape. This may be the outer shape of the patch, which itself may be made up of individual dots of adhesive or thin stripes of adhesive. In particular, the crotch adhesive patches have a length in the longitudinal direction (y) in a range from 10 to 50 mm, preferably from 30 to 46 mm and a width in the transverse direction (x) in a range from 4 to 20 mm, preferably from 8 to 15 mm.

The rear adhesive patches may have a length in the longitudinal direction (y) in a range from 10 to 50 mm, preferably from 20 to 30 mm and a width in the transverse direction (x) in a range from 4 to 20 mm, preferably from 8 to 15 mm. In an embodiment, the crotch adhesive patches may have a length in the longitudinal direction (y) that is greater than a length in the longitudinal direction (y) of the rear adhesive patches. The shorter rear adhesive patches may then better be able to fit within the area of the rear flaps, despite their offset.

The crotch adhesive patches and the rear adhesive patches may be arranged essentially parallel to the central longitudinal axis (Y). This will generally be the case when the adhesive patches are applied using slot coating technology in a process that has a process direction aligned with the longitudinal axis. Furthermore, the adhesive patches may each be spaced a spacing distance from the central longitudinal axis (Y) and a spacing distance d1 of the first crotch adhesive patch is equal to a spacing distance d2 of the first rear adhesive patch and a spacing distance d3 of the second crotch adhesive patch is equal to a spacing distance d4 of the second rear adhesive patch. By aligning the front and rear adhesive patches, a relatively narrow carrier web can be used for each of the respective left and right sets of adhesive patches and a single slot coater can be used.

In certain embodiments the spacing distances d1 and d2 may be different from the spacing distances d3 and d4. In other words, the left and the right adhesive patches may not be positioned at the same transverse position on the article. This may be preferred or required in order to best locate the adhesive patches with respect to the respective wings.

The first and second rear flaps may also have any appropriate size and form according to the intended user and garment. The flaps extend outwardly in the transverse direction a distance dF from the central longitudinal axis (Y) and the first and second front wings extend outwardly a distance dW. In preferred embodiments, dF is equal to or greater than dW.

The position of all of the adhesive patches is preferably a sufficient distance from an edge of the absorbent article. It is generally undesirable that an adhesive patch is placed outside the contour of the article during manufacture, even allowing for tolerances. According to certain methods of manufacture, the outer contour may be cut, including any outwardly extending portion of the adhesive patch. Nevertheless, it may be undesirable to have a cut edge of the adhesive patch coinciding with an edge of the article as this may lift or peel during use. Preferably, a minimum distance between any of the adhesive patches and an outer contour of the absorbent article is 5 mm, preferably 10 mm.

As mentioned above, each of the adhesive patches may be applied using a carrier web. Preferably, the carrier web is in the form of a release material, such as siliconized paper, that remains on the adhesive patch on completion of manufacture as a release cover. A single release cover may cover all of the adhesive patches. Alternatively, left and right release covers may cover the respective left and right adhesive patches. More preferably, individual release covers may be present on each of the adhesive patches.

The rear flaps may extend transversally outwards but may also extend rearwardly. In an embodiment, this results in the rear edge of the absorbent article having a cut-out between the respective rearmost extensions of the first and left rear flaps. The cut-out may have an arcuate shape extending between the first rear flap and the second rear flap. In an embodiment, the rear cut-out has a maximum depth (dR) with respect to a line joining a rearmost point of the first and second rear flaps (XR) in a range from 10 to 20 mm, preferably from 12 to 15 mm. Additionally or alternatively, the rear cut-out has the shape of a shallow isosceles triangle, forming angles with respect to the central longitudinal axis (Y) in a range of 18 to 30 degrees, preferably from 20 to 22 degrees. The presence of the cut out may be advantageous in removing excess material from between the buttocks regions. This can improve the comfort during use and increase the pliability of the product towards the body of a user. Additionally, it can allow for efficient cutting-out of the absorbent articles if the shape of the cut-out matches in shape the front edge of the front portion.

The invention also relates to a method of manufacturing an absorbent article, including:
transporting a continuous precursor web in a longitudinal direction, the continuous precursor web including at least a backsheet and an absorbent core and having left and right lateral edges;
providing a continuous carrier web structure;
repeatedly applying front and rear adhesive patches to respective left and right sides of the carrier web structure, wherein the front left adhesive patch is offset from the front right adhesive patch and the rear left adhesive patch is offset from the rear right adhesive patch in the longitudinal direction;
bringing the carrier web structure and the precursor web together to connect the adhesive patches to the precursor web; and
cutting out individual absorbent articles from the precursor web, the individual articles having wings and wherein adhesive patches are located on the wings. The absorbent article may be an article as defined above or hereinafter.

In certain embodiments, the carrier web structure includes left and right carrier webs and the front left adhesive patch and the rear left adhesive patch are provided on the left carrier web and the front right adhesive patch and the rear right adhesive patch are provided on the right carrier web. This allows for less material usage, since the carrier web may be limited to the respective widths of the adhesive patches.

In a particular embodiment, the carrier web is a release paper and the method includes cutting the carrier web into individual release papers, each carrying a respective adhesive patch, prior to connecting the patches to the precursor web. Cutting may take place by conventional means e.g. by rotating knives against a transfer drum, whereby the transfer drum also acts to connect the adhesive patches together with their respective individual release papers to the respective locations on the precursor web.

In one embodiment, a distance between successive adhesive patches is constant. This means not only that the distance between the front and rear adhesive patches is the same for both the left and the right side but also that it is the same as the distance between the rear adhesive patch on a first absorbent article and the front adhesive patch on a subsequent article. Such an arrangement simplifies the synchronisation between the adhesive applicators and the transport arrangements for the respective webs. This is particularly desirable for the case that the adhesive patches are applied by slot coating of a liquid adhesive material.

Cutting out individual absorbent articles from the precursor web may take place with or without material loss between a front edge and a rear edge of adjacent articles.

### BRIEF DESCRIPTION OF THE DRAWINGS

The features and advantages of the present disclosure will be appreciated upon reference to the following drawings of a number of exemplary embodiments, in which:

FIG. 1A shows a plan view of a sanitary napkin according to an embodiment of the invention;

FIG. 1B shows a plan view of the sanitary napkin of FIG. 1A, illustrating certain dimensions;

FIG. 2 shows a transverse cross-section through the napkin of FIG. 1A taken at the central transverse axis X; and

FIG. 3 shows a schematic illustration of a machine for manufacturing absorbent articles according to the invention.

### DESCRIPTION OF ILLUSTRATIVE EMBODIMENTS

Different aspects of the present disclosure will be described more fully hereinafter with reference to the enclosed drawings. The embodiments disclosed herein can, however, be realized in many different forms and should not be construed as being limited to the aspects set forth herein.

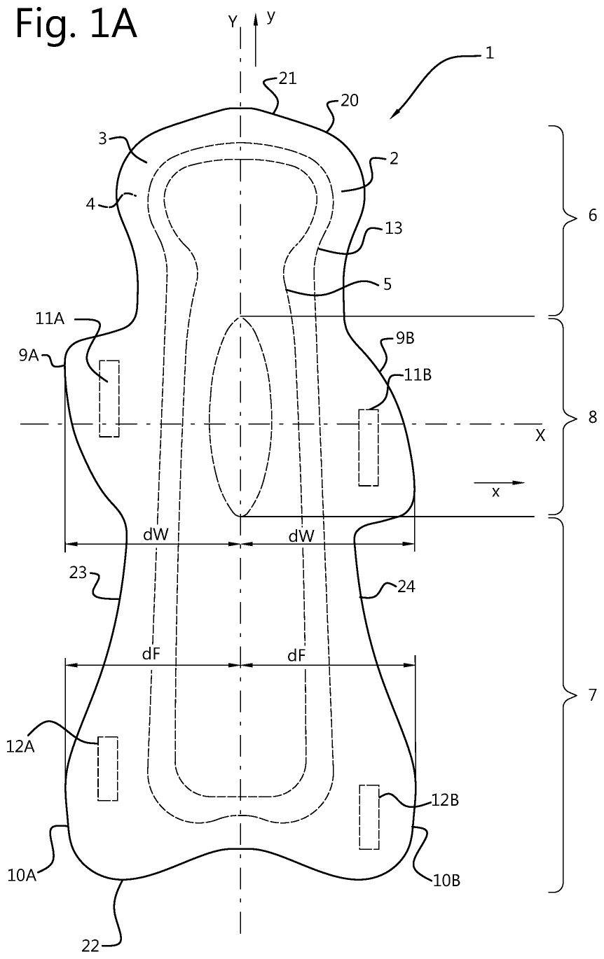

With initial reference to FIG. 1A, there is shown a plan view onto the topsheet of an absorbent article in the form of a sanitary napkin 1 manufactured in accordance with an embodiment of the invention. The sanitary napkin 1 is based on an absorbent structure for absorbing body exudates from a wearer in order to provide a dry, comfortable and odour-free feeling for the wearer.

From FIG. 1A it can be understood that the sanitary napkin 1 includes a main body 2 with a liquid-permeable topsheet 3 and a liquid-impermeable backsheet 4. The sanitary napkin 1 also includes an absorbent core 5 which is sandwiched between the topsheet 3 and the backsheet 4. The topsheet 3 is arranged at the surface of the sanitary napkin 1, i.e. the side facing the wearer. The backsheet 4 is arranged at the underside of the sanitary napkin 1, i.e. the side facing an undergarment (not shown) of the wearer. Furthermore, both the topsheet 3 and the backsheet 4 extend laterally outside of the absorbent core 5 along the whole perimeter 20 of the main body 2.

In the illustrated embodiment, an acquisition layer 13 is arranged between the absorbent core 5 and the topsheet 3. The acquisition layer 13 can for example be in the form of an airlaid layer, a spunlace layer, a high-loft, foam or any other type of material layer which may be used in an absorbent article in order to act as a liquid acquisition and absorption layer. The acquisition layer 13 is adapted to quickly receive and temporarily store discharged liquid before it is absorbed by the absorbent core 5. Such acquisition layer 13 may be composed of for example airlaid nonwoven, spunlace nonwoven, high loft nonwoven or foam materials. An airlaid nonwoven can be produced with fluff, wood pulp, and here the fluff fibres are dispersed into a fast moving air stream and condensed onto a moving screen by means of pressure and vacuum. The web can be bonded with resin and/or thermal plastic resin dispersed with the pulp. The web can be thermobonded (by heat), latex bonded (by adhesive) or multibonded (a combination of thermos and latex bonding) or mechanically bonded (high compression and temperature, bonding by hydrogen). The grammage of the airlaid nonwoven may be for example from 50 to 100 gsm.

The topsheet 3, backsheet 4 and the absorbent core 5 may consist of any materials suitable for their particular purposes, as will be discussed in further detail below. Also, the above-mentioned layers 3, 4, 5 may be connected to each other by any conventional means such as by means of an adhesive, heat bonding or ultrasonic bonding.

Furthermore, and although not shown in the drawings, the sanitary napkin 1 can be configured in a manner having different material layers, densities or material components as seen along a horizontal plane of the sanitary napkin 1, i.e. in addition to being configured with different layers in the vertical direction.

As shown in FIG. 1A, the sanitary napkin 1 can be divided into a front portion 6, a back portion 7 and a crotch portion 8. The front portion 6 is intended to be oriented in a direction towards the wearer's belly during use of the sanitary napkin 1. Furthermore, the sanitary napkin 1 has a longitudinal extension in a longitudinal direction y along a central longitudinal axis Y from a front edge 21 in the front portion 6 to a rear edge 22 in the rear portion 7. It also has a transverse extension in a transverse direction x, along a central transverse axis X between a left side edge 23 and a right side edge 24.

Furthermore, according to the embodiment, the sanitary napkin 1 is formed with two front wings 9A, 9B, which are configured for fastening the napkin 1 to an undergarment of the wearer. More precisely, the sanitary napkin 1 includes a first front wing 9A and a second front wing 9B, each of which extends outwardly from the main body 2 and in opposite directions. The wings 9A, 9B extend generally along the direction of the transversal axis X. Also, the wings 9A, 9B are provided with crotch adhesive patches 11A, 11B which are configured for fastening the wings 9A, 9B to the undergarment when the wings 9A, 9B are folded under the main body 2 in a direction towards the longitudinal axis Y. It will be noted that the crotch adhesive patches 11A, 11B are provided on the underside of the napkin 1, connected to the backsheet 4 and shown only in shadow.

As indicated in FIG. 1A, the two wings 9A, 9B are arranged in an asymmetrical manner with respect to the longitudinal axis Y. Both wings are located within the crotch portion 8 but the first front wing 9A is located closer to the front portion 6, while the second front wing 9B is located closer to the rear portion 9B. Such wings and their advantages are described for example in WO2019/008091, the contents of which are enclosed herein in their entirety.

The sanitary napkin 1 is further provided with two rear flaps 10A, 10B, which can also be fastened to an undergarment of the wearer. These include first rear flap 10A and second rear flap 10B. The rear flaps 10A, 10B extend outwardly and rearwardly from the main body 2. The flaps 10A, 10B are provided with rear adhesive patches 12A, 12B also located on the backsheet 4 side of the napkin 1.

The crotch adhesive patches 11A, 11B and the rear adhesive patches 12A, 12B are located on their respective wings 9A, 9B and flaps 10A, 10B at positions that ensure that they are spaced from the periphery 20. In particular, the adhesive patches are each spaced a spacing distance d from the central longitudinal axis (Y). The first crotch adhesive patch 11A is at a spacing distance d1. The first rear adhesive patch 12A is at a spacing distance d2. The second crotch adhesive patch is at a spacing distance d3 and the second rear adhesive patch 12B is at a spacing distance d4. In the illustrated embodiment, the first crotch adhesive patch 11A and the first rear adhesive patch 12A are aligned in the longitudinal direction x with d1 equal to d2 and d3 equal to d4. Furthermore, in the illustrated embodiment, d1 is equal to d3 and it will thus be understood that all of the adhesive patches are equally spaced from the central longitudinal axis Y.

FIG. 1B is a similar view of the sanitary napkin 1 to that of FIG. 1A and is used to more clearly illustrate respective dimensions and positions of certain items. As can be seen, the first and second crotch adhesive patches 11A, 11B are offset with respect to each other. The first crotch adhesive patch 11A is located an offset distance dO further towards the front edge 21 than the second crotch adhesive patch 11B. It will be noted that the offset distance dO ensures that the first crotch adhesive patch 11A falls within the first front wing 9A, while the second crotch adhesive patch 11B falls within the second front wing 9B. Both the first crotch adhesive patch 11A and the second crotch adhesive patch 11B are distanced by a minimum spacing dM from the perimeter 20.

The first and second rear adhesive patches 12A, 12B are also offset with respect to each other by the same offset distance dO, whereby the second rear adhesive patch 12B is closer to the rear edge 22. In this case, it is noted that the rear flaps 10A, 10B are symmetrical about the longitudinal axis Y. Nevertheless, the area of the rear flaps 10A, 10B is sufficient that the rear adhesive patches 12A, 12B may be positioned without approaching the perimeter 20, being distanced from the perimeter by at least the minimum spacing dM.

Between the rear flaps 10A, 10B, the rear edge 22 is provided with a cut-out 30 having an arcuate shape. The cut-out 30 corresponds generally to the shape of the front edge 21 as will be explained further below. The rear cut-out 30 has a maximum depth dR with respect to a baseline XR joining a rearmost point of the first and second rear flaps 10A, 10B. The rear cut-out 30 has generally the shape of a shallow isosceles triangle, forming angles a with respect to the central longitudinal axis (Y) of around 20 degrees.

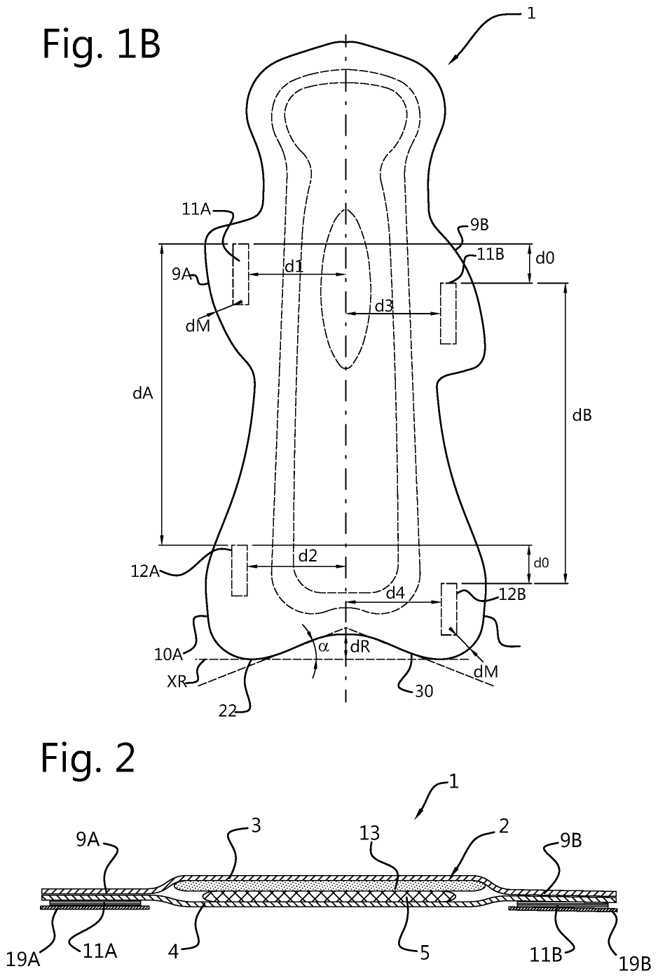

FIG. 2 is a cross-sectional view at the transverse axis X shown in FIG. 1, looking forwards. The absorbent core 5 is sandwiched between the topsheet 3 and the backsheet 4. As mentioned above, an acquisition layer 13, is arranged between the absorbent core 5 and the topsheet 3. Also, the two wings 9A, 9B are shown, provided with adhesive patches 11A 11B, applied on their undersides i.e. on the side which does not face the wearer.

Not visible in FIGS. 1A, 1B but shown in FIG. 2, the adhesive patches 11A, 11B are covered with a release paper layer 19A, 19B, which is present when the sanitary napkin 1 is in its non-used condition. When the sanitary napkin 1 is to be used, the release paper layers 19A, 19B are removed by the user so that the sanitary napkin 1 can be fastened to the undergarment.

The properties and design of the various layers and components which form part of the sanitary napkin 1 will now be described more in detail, with reference primarily to FIGS. 1 and 2.

According to an embodiment, the topsheet 3 is formed by a fluid permeable nonwoven fabric or film which is made of thermoplastic synthetic fibers. The topsheet 3 is sufficiently liquid-permeable to allow discharged body fluids to penetrate through the thickness of the topsheet 3. Also, the topsheet 3 is suitably manufactured from a material which is compliant and soft-feeling to the skin of the wearer.

According to different embodiments, the topsheet 3 may be manufactured from various web materials such as for example nonwoven films, foams, or combinations of the above-mentioned materials. The topsheet 3 may also be perforated.

Furthermore, the backsheet 4 is constituted by a liquid-impermeable and breathable layer such as a polymeric film, for example a film of polyethylene or polypropylene. According to different embodiments, the materials which can be used for the backsheet 4 include thin and flexible fluid impermeable plastic films, or fluid impermeable nonwoven materials, fluid impermeable foams and fluid impermeable laminates.

According to the embodiment shown in the drawings, the backsheet 4 is formed by a single layer, but can alternatively be formed by a multi-layered structure, i.e. a laminate, wherein at least one layer is fluid impermeable. Furthermore, the backsheet 4 can optionally be elastic in any direction. Also, backsheet materials which are not fully liquid impermeable but only resistant to fluid penetration may be used, particularly in cases where relatively small amounts of body exudates are expected to be absorbed by the sanitary napkin 1.

According to further embodiments, the backsheet 4 may be breathable, implying that air and vapor may pass through the backsheet 4. Furthermore, the backsheet 4 may optionally have an outer, garment-facing surface of a textile material such as nonwoven

The absorbent core 5 may be formed by one or more layers including fibres of cellulosic fluff pulp. According to alternative embodiments, the absorbent core 5 can be made up of any suitable absorbent or fluid-absorbing material as known in the art, for example foam, fiber waddings and similar materials. As mentioned above, different types of material layers can be used, alone or in combination, for example airlaid, spunlace and highloft material, depending on the required properties and the field of use of the sanitary napkin 1 in question.

According to one embodiment, the absorbent article 1 is a liner, in which case the absorbent core 5 may only be made of one thin material, for example a nonwoven layer.

According to a further embodiment, the absorbent core 5 includes a suitable amount of superabsorbent particles. Such superabsorbent material is well known in the field of absorbent articles, and is constituted by a water-swellable and water-insoluble material which is capable of absorbing large quantities of fluid upon formation of a hydrogel. Normal superabsorbent materials are capable of absorbing fluids of at least 10 times its own weight. According to an embodiment, the amount of said superabsorbent particles is at least 10% by weight, i.e. in relation to the total weight of the absorbent core 5.

The superabsorbents are mixed into the material of the absorbent core 5. The absorbent core 5 may further incorporate components for improving the properties of the absorbent core 5. Some examples of such components are binder fibers, fluid-dispersing materials, fluid acquisition materials, etc. as known in the art. According to an embodiment, a mixture of cellulose fluff pulp and superabsorbent articles is mixed in a generally homogeneous manner throughout the entire absorbent core 5.

According to further embodiments, the absorbent core 5 may be a homogeneous structure or may be a layered structure with laminates of the same or different materials. The absorbent layers may have uniform thickness or may vary in thickness in different parts of the layers. Also, the basis weight and composition of the absorbent core 1 may vary within such absorbent layers.

Furthermore, as known by the skilled person, the various layers of the sanitary napkin 1 can suitably be attached by means of layers of adhesive material. Such adhesive layers are not shown in the drawings.

As regards the adhesive patches 11A, B and 12A, B The adhesive material used can be any of a large number of pressure sensitive adhesives that are commercially available, including cold pressure-sensitive adhesives such as the acrylate adhesives, generally combined with tackifiers such as polyterpenes, or rapid-setting thermoplastic adhesives (or hot-melt adhesives) such as styrene and butadiene copolymers. Other ingredients such as fillers, antioxidants and pigments may be included. The adhesive patches may also include a double faced adhesive tape or another type of adhesive. The adhesive may be applied to the backsheet in various patterns, such as for example spots, squares, single full or intermittent lines or multiple full or intermittent lines, which may be parallel or not. Other patterns are also possible. Suitably, the pressure sensitive adhesive is a pressure sensitive hot-melt adhesive. The adhesive can be spray-coated or slot-coated to the garment facing surface of the backsheet in the respective wing or flap region or applied first to the release paper layers 19A, 19B as will be described further below.

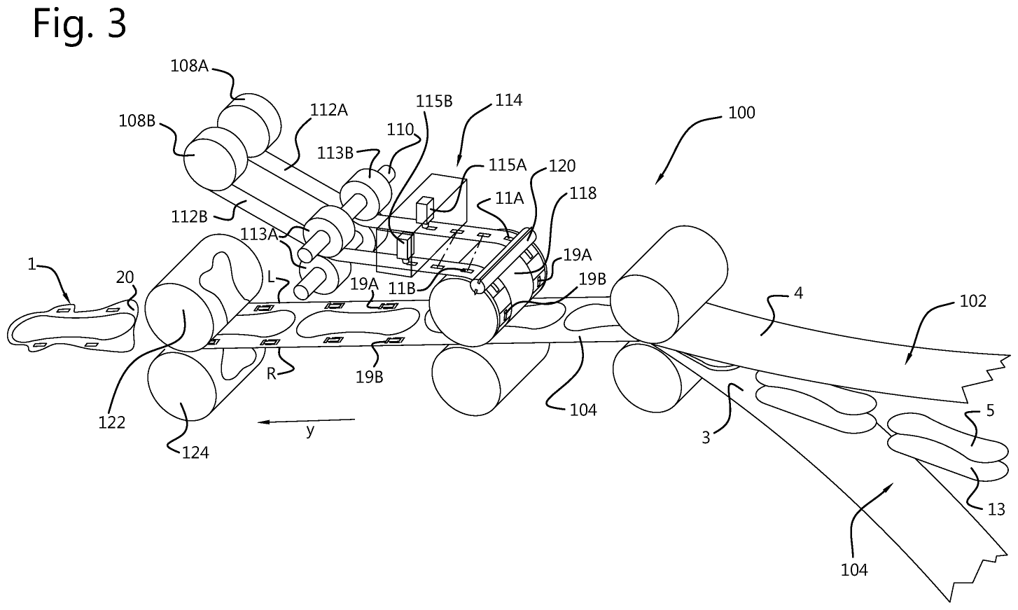

FIG. 3 is a schematic illustration of a machine 100 for manufacture of absorbent articles according to the invention such as the napkins 1 described in FIGS. 1 and 2 above.

The machine 100 includes a primary transport path 102, which delivers a precursor web 104 in a process direction y, corresponding to a longitudinal direction of the napkins 1. The precursor web 104 includes a topsheet 3 a backsheet 4, with cores 5 and acquisition layers 13 previously assembled together. The assembly of these elements is generally conventional and not further described as it does not form a part of the present invention.

The machine 100 further has left and right carrier rolls 108A, 108B, which deliver left and right carrier webs 112A, 112B of release paper to slot coating station 114. The speed of delivery is controlled by left and right feeder rollers 113A, 113B which are mounted to rotate on a common shaft 110. At slot coating station 114, left and right slot coating nozzles 115A, 115B deliver adhesive material to the left and right carrier webs 112A, 112B. The left and right slot coating nozzles 115A, 115B are arranged to deliver the adhesive material with an offset such that adhesive material is delivered to the left carrier web 112A in advance of the right carrier web 112B as offset adhesive patches 11A, 11B and 12A, 12B. The left and right carrier webs 112A, 112B proceed to a transfer drum 118, where the carrier webs 112A, 112B are cut by rotating knives 120 into individual release paper layers 19A, 19B each carrying a respective adhesive patch 11A, 11B. The transfer drum 118 is a conventional vacuum drum that retains the release paper layers 19A, 19B on its surface by suction once they have been cut. It also acts as an anvil against which the rotating knives 120 can cut.

On further rotation, the transfer drum 118 brings the adhesive patches 11A, 11B into engagement with the precursor web 104 whereby alignment provisions ensure that the adhesive patches 11A, 11B are correctly aligned with areas on the precursor web 104 designated to become the front wings 9A, 9B.

Subsequently, the precursor web 104 passes over vacuum drum 124, which engages with outer contour cutter 122 to cut around the perimeter 20 of the main body 2 to form the napkin 1.

Thus, the present disclosure has been described by reference to the embodiments discussed above. It will be recognized that these embodiments are susceptible to various modifications and alternative forms well known to those of skill in the art without departing from the spirit and scope of the invention as defined by the appended claims.

1. An absorbent article comprising a liquid-permeable topsheet, a liquid-impermeable backsheet, an absorbent core arranged between the topsheet and the backsheet, a central transverse axis (X) and a central longitudinal axis (Y), wherein the absorbent article comprises a front portion, a rear portion, and a crotch portion arranged between the front and rear portions, and a length of the absorbent article in the longitudinal direction (y) is defined between a front edge of the front portion and a rear edge of the rear portion at the central longitudinal axis, the crotch portion comprising first and second front wings that extend outwardly from the crotch portion in the transverse direction (x), the rear portion comprising first and second rear flaps that extend outwardly from the rear portion in the transverse direction (x), characterized in that the first and second front wings each comprise respective first and second crotch adhesive patches, wherein the crotch adhesive patches are offset from each other in the longitudinal direction (y) by an offset distance dO, and the first and second rear flaps each comprise respective first and second rear adhesive patches, wherein the rear adhesive patches are also offset from each other in the longitudinal direction (y) by the same offset distance.

2. The absorbent article of claim 1, wherein a repeat distance (dR) between the first crotch adhesive patch and the first rear adhesive patch and between the second crotch adhesive patch and the second rear adhesive patch in the longitudinal direction (y) is in a range from 100 mm to 250 mm, preferably from 140 mm to 200 mm.

3. The absorbent article of claim 1, wherein the offset distance (dO) is in a range of from 10 mm to 40 mm, preferably between 15 mm and 30 mm, more preferably around 20 mm.

4. The absorbent article of claim 1, wherein the first and second front wings extend outwardly from the crotch portion in an asymmetric manner with respect to the central longitudinal axis (Y) and also preferably with respect to the central transverse axis (X).

5. The absorbent article of claim 1, wherein the first and second rear flaps are symmetrical with respect to the central longitudinal axis (Y).

6. The absorbent article of claim 1, wherein the absorbent core has a smaller extension in a plane of the longitudinal and transverse axes than an extension of the backsheet and/or topsheet, the body facing surface of the backsheet preferably being joined at a perimeter to the garment facing surface of the topsheet.

7. The absorbent article of claim 1, wherein the backsheet and the topsheet have substantially the same extension in a plane of the longitudinal and transverse extension of the absorbent article and edge areas of the absorbent article are joined together by means of embossing and/or ultrasonic processing.

8. The absorbent article of claim 1, wherein the crotch adhesive patches and the rear adhesive patches are generally rectangular in shape.

9. The absorbent article of claim 1, wherein the crotch adhesive patches have a length in the longitudinal direction (y) that is greater than a length in the longitudinal direction (y) of the rear adhesive patches, preferably being in a range from 10 to 50 mm, preferably from 30 to 46 mm and a width in the transverse direction (x) in a range from 4 to 20 mm, preferably from 8 to 15 mm.

10. The absorbent article of claim 1, wherein the rear adhesive patches have a length in the longitudinal direction (y) in a range from 10 to 50 mm, preferably from 20 to 30 mm and a width in the transverse direction (x) in a range from 4 to 20 mm, preferably from 8 to 15 mm.

11. The absorbent article of claim 1, wherein the crotch adhesive patches and the rear adhesive patches are arranged essentially parallel to the central longitudinal axis (Y).

12. The absorbent article of claim 1, wherein the adhesive patches are each spaced a spacing distance d from the central longitudinal axis (Y) and a spacing distance d1 of the first crotch adhesive patch is equal to a spacing distance d2 of the first rear adhesive patch and a spacing distance d3 of the second crotch adhesive patch is equal to a spacing distance d4 of the second rear adhesive patch.

13. The absorbent article of claim 12, wherein the spacing distances d1 and d2 are different from the spacing distances d3 and d4.

14. The absorbent article of claim 1, wherein the first and second rear flaps extend outwardly in the transverse direction a distance dF from the central longitudinal axis (Y) and the first and second front wings extend outwardly a distance dW from the central transverse axis (Y) and dF is equal to or greater than dW.

15. The absorbent article of claim 1, wherein a minimum distance between an adhesive patches and an outer contour of the absorbent article is 5 mm, preferably 10 mm.

16. The absorbent article of claim 1, wherein each of the adhesive patches is provided with a respective release cover.

17. The absorbent article of claim 1, wherein the length in the longitudinal direction is in a range from 150 to 400 mm, preferably from 280 to 320 mm

18. The absorbent article of claim 1, further comprising a liquid acquisition sheet arranged between the topsheet and the absorbent core.

19. The absorbent article of claim 1, wherein the rear edge is provided with a cut-out having an arcuate shape located between the first rear flap and the second rear flap.

20. The absorbent article of claim 1, wherein the front and crotch portions each comprise 30% of a total length of the absorbent article in the longitudinal direction (y), and the rear portion comprises 40% of the total length.

21. A method of manufacturing an absorbent article, comprising:
transporting a continuous precursor web in a longitudinal direction, the continuous precursor web comprising at least a backsheet and an absorbent core and having left and right lateral edges;
providing a continuous carrier web structure;
repeatedly applying front and rear adhesive patches to respective left and right sides of the carrier web structure, wherein the front left adhesive patch is offset from the front right adhesive patch and the rear left adhesive patch is offset from the rear right adhesive patch in the longitudinal direction;
bringing the carrier web structure and the precursor web together to connect the adhesive patches to the precursor web; and
cutting out individual absorbent articles from the precursor web, the individual articles having wings and wherein adhesive patches are located on the wings

22. The method according to claim 21, wherein the carrier web structure comprises left and right carrier webs and the front left adhesive patch and the rear left adhesive patch are provided on the left carrier web and the front right adhesive patch and the rear right adhesive patch are provided on the right carrier web.

23. The method according to claim 21, wherein the carrier web is a release paper and the method comprises cutting the carrier web into individual release papers, each carrying a respective adhesive patch, prior to connecting the patches to the precursor web.

24. The method according to claim 21, wherein a distance between successive adhesive patches is constant.

25. The method according to claim 21, wherein the adhesive patches are applied by slot coating of a liquid adhesive material.

26. The method according to claim 21, wherein the absorbent article includes a liquid-permeable topsheet, a liquid-impermeable backsheet, an absorbent core arranged between the topsheet and the backsheet, a central transverse axis (X) and a central longitudinal axis (Y), wherein the absorbent article comprises a front portion, a rear portion, and a crotch portion arranged between the front and rear portions, and a length of the absorbent article in the longitudinal direction (y) is defined between a front edge of the front portion and a rear edge of the rear portion at the central longitudinal axis, the crotch portion comprising first and second front wings that extend outwardly from the crotch portion in the transverse direction (x), the rear portion comprising first and second rear flaps that extend outwardly from the rear portion in the transverse direction (x), characterized in that the first and second front wings each comprise respective first and second crotch adhesive patches, wherein the crotch adhesive patches are offset from each other in the longitudinal direction (y) by an offset distance dO, and the first and second rear flaps each comprise respective first and second rear adhesive patches, wherein the rear adhesive patches are also offset from each other in the longitudinal direction (y) by the same offset distance.
